(19) United States
(12) Patent Application Publication
(10) Pub. No.: US 2025/0290906 A1
(43) Pub. Date: Sep. 18, 2025

(54) Food Quality Indicator

(71) Applicant: Innoscentia AB, Lund (SE)

(72) Inventor: Rambabu Atluri, Malmö (SE)

(21) Appl. No.: 18/859,716

(22) PCT Filed: May 2, 2023
(86) PCT No.: PCT/EP2023/061469

§ 371 Date: Oct. 24, 2024

(30) Foreign Application Priority Data
May 3, 2022 (SE) 2250537-4

(51) Int. Cl. G01N 33/00; G01N 1/22; G01N 33/02

(52) U.S. Cl. CPC G01N 33/0062; G01N 1/2226; G01N 33/02; G01N 2001/2232

(57) ABSTRACT

A food quality indicator including an ink layer configured to detect and indicate the presence of analytes, such as volatile organic compounds (VOC), an adhesive layer, wherein the adhesive layer is a double-sided adhesive layer, and a semi-permeable layer that is permeably to gases and impermeable to liquids. The ink layer including of a reactive ink absorbed on a porous substrate and is enclosed between the adhesive layer and the semi-permeable layer. The semi-permeable may further include a coated protective layer configured to prevent the migration of ink from the ink layer. A method for preparing the ink and the protective layer of the food quality indicator as well as the food quality indicator are provided.

### CROSS-REFERENCE TO RELATED APPLICATIONS

This application is the United States national phase of International Patent Application No. PCT/EP2023/061469 filed May 2, 2023, and claims priority to Swedish Patent Application No. 2250537-4 filed May 3, 2022, the disclosures of which are hereby incorporated by reference in their entireties.

### BACKGROUND OF THE INVENTION

### Field of the Invention

The present invention relates in general to the field of food quality indicators and manufacturing of said food quality indicators. The present invention further relates to a food packaging comprising said food quality indicators.

### Description of Related Art

Many food articles, such as meat, poultry, fish, dairy products, fruits vegetables, processed food are perishable and it can be difficult for customers to detect when a particular food has expired, especially if the food is enclosed in a package.

Several perishable food articles are packaged in food trays using barrier films. Poultry is for instance often provided in a food tray with a top lidding barrier film. Such food article packaging is provided for a number or reasons, namely for sanitary reasons, ensuring a safe delivery of the product from the manufacturer to the customer, increasing the shelf life of the product.

The term barrier film indicates the function of the film to seal the food product from the outside and to preserve the original conditions inside the package. Thus, the penetration of gases that have an impact on the quality of the food products, such as oxygen, carbon dioxide, nitrogen, and water vapor among others, into the food package are prevented.

Today, an expiry date is provided on food packages to give the customer an indication and understanding of the lifespan of a food product and is a basic safety requirement. Food expiry dates are generally set during the product development phase and the calculations are based on certain assumptions, such that the perishable material is stored under certain conditions, such as refrigerated. Thus, if the food has not been stored properly, for instance because of a broken refrigerator or perhaps a faulty packaging, the expiry date may not apply. However, in some cases where the food has been kept under the right conditions, the food may still be comestible past the recommended expiry date provided on the package. This could result in edible food being thrown away and thus additional food waste. There is an unconditional trust on “best before dates” or “use by date”, which does not reflect the conditions of manufacturing, packaging and storage throughout the supply chain.

Many food products, such as meat, pork, poultry, processed food, produce analytes upon decomposition.

In meat in particular, analytes are produced through lipid oxidation, through Maillard reaction or Strecker degradation, which are responsible for meat flavor development. The analytes that are generated during meat storage can have an olfactory impact and lead to the food being inedible. A large number of analytes produced depend on the storage conditions and type of packing. The most common analytes are volatile organic compounds (VOCs) such as include Alcohols, Amines, Aldehydes, Ketones, Esters and Sulphides and volatile amines also called Biogenic amines (BA) such as ethylamine and methylamines.

The lack of real-time monitoring labels from farm to fork in the individual packaging increase the risk of consuming the low-quality food and food wastage.

Intelligent packaging or “smart packaging” have been developed to address the problem by developing colorimetric sensor arrays, gas sensors, time-temperature sensors, pH indicators, to name a few. Unfortunately, most of them did not make it into the market due to complications in the result analysis, difficulties attaching the sensor/measuring means to the food packaging, cost, and regulatory barriers of the components.

Colour indicators printed on a package, where the indicator changes colour to indicate that the food has expired is a promising solution. However, providing a single indicator that can detect multiple analytes has been a challenge. The difficulty to print a colour changing ink on a surface of the food package has also restricted previous solutions.

Hence, there is a need for a reliable food freshness indicator which address the disadvantages and shortcomings of the prior art, which can be manufactured in a cost-efficient manner.

### SUMMARY OF THE INVENTION

Accordingly, the present invention is to solve or at least mitigate the problems related to prior art. This object is achieved by means of the technique set forth in the appended independent claims; preferred embodiments being defined in the related dependent claims.

According to a first aspect, a food quality indicator is provided. The food quality indicator has an ink layer including an ink absorbed on a porous substrate. The ink of the ink layer is configured to indicate the presence of analytes. The food quality indicator also has an adhesive layer which is a double-sided adhesive layer comprising a first side and a second side. The food quality indicator also has a semi-permeable layer with a first surface and a second surface, the semi-permeable layer is permeable to gases and impermeable to liquids. The ink layer is arranged between the second side of the adhesive layer and the first surface of the semi-permeable layer.

An advantage of providing an ink layer configured to indicate the presence of analytes such as volatile organic compounds (VOCs), volatile gases, volatile amines, biogenic amines, or volatile sulphur compounds is that the end consumer is provided with simple and cheap yet accurate means for continuously monitoring the quality of a food product. The food quality indicator successively changes colour when detecting the analytes, thus the consumer is made aware of the lifespan of the food product. Such that the consumer will know if a food product has perished or is still edible.

The ink layer is made by absorbing ink on a porous substrate having functions of absorbing either water-based or solvent based reactive ink and also preventing the migration of the reactive ink into the food product.

Moreover, by providing the ink layer enclosed between the adhesive layer and the semi-permeable layer, the ink is protected from its surroundings and it will take some time for the analytes to reach the ink layer. This is an advantageous feature, as the food product will not perish directly upon generating analytes. The analytes can only access the ink layer by passing though the semi-permeable layer. Thus, a controlled migration of analytes from the food product to the semi-permeable layer is achieved.

According to an embodiment, the analytes have to migrate through the semi-permeable layer of the food quality indicator to reach the ink layer.

Thus, a controllable and measurable migration is obtained.

According to one embodiment, the food quality indicator further includes a protective layer that is coated on the first surface of the semi-permeable layer. The protective layer is configured to prevent the migration of ink from the ink layer.

An advantage of preventing the migration of ink with a protective layer, is that the food product being monitored by the food quality indicator is further protected from being in contact with the ink. Furthermore, it is not desirable to have an ink migrating from the food quality indicator as this could affect the performance of the ink. The ink may not change colour as efficiently upon entering in contact with analytes if the ink is smudged out. Moreover, it may be difficult for the user to read the monitoring result from the analytes. It is thus desirable to keep the ink in the area enclosed between the adhesive layer and the semi-permeable layer for providing clear and accurate results. The analytes can only access the ink layer by passing though the semi-permeable layer and the protective layer. Thus, a controlled migrating of analytes from the food product to the semi-permeable layer and the protective layer is achieved.

According to one embodiment, the protective layer is permeable to gases. Thus, permitting the entry of analytes such as volatile organic compounds (VOCs), volatile gases, volatile amines, biogenic amines, or volatile sulphur compounds.

According to one embodiment, the analytes migrate through the semi-permeable layer and the protective layer to reach the ink layer.

According to one embodiment, the protective layer is a hydrophobic layer. The protective layer being hydrophobic will result in the ink being repelled by the protective layer. As the protective layer is coated inside the semi-permeable layer and the ink layer is enclosed between the adhesive layer and the semi-permeable layer, the ink layer will be kept in this area.

According to one embodiment, the protective layer comprises a polymer and a solvent.

According to one embodiment, the polymer and the solvent of the protective layer have a weight ratio in the interval 0.01:2 to 0.2:0.4, such as 0.01:1, preferably 0.15:0.56.

According to one embodiment, the polymer of the protective layer is selected from a group consisting of polydimethylsiloxane (PDMS), polyurethane, polyamide or polyethylene, polyvinyl alcohol (PVA), polyvinyl chloride (PVC), ethyl cellulose (EC), methyl cellulose (MC), hydroxypropyl methylcellulose (HPMC), or any combinations thereof with a weight ratio between 0.1 to 0.5.

According to one embodiment, the solvent of the protective layer is selected from a group of solvents having a viscosity between 20 to 300 cP and a boiling point between 34.6° C. to 190° C., more preferably between 50 to 90° C., wherein the solvents have vapour pressures between 2.9 kPa to 170.6 kPa, preferably between 21.6 kPa to 110.6 kPa.

An advantage of the protective layer provided according to the present method is that it is hydrophobic and permeable to gases. Most substrates are highly porous substrates which are permeable to both liquids and gases. Achieving a selective diffusion on permeable and highly permeable substrates is a challenge in particular when it comes to differentiating between gases and liquids which is overcome by the protective layer of the present invention.

According to one embodiment, the semi-permeable layer is non-transparent.

An advantage of the semi-permeable layer being non-transparent is that it provides a background with contrast for the ink layer and the other parts of the food quality indicator so that colour changes and results are easy to read and clear to the end customer. Since the food quality indicator is to be provided inside a food package it is important that the results are easy to perceive through the food package.

According to one embodiment, the semi-permeable layer is configured to be printed on.

An advantage of the semi-permeable layer being a material that is printable on, is that additional information such as a text area can be provided on the food quality indicator to give the customer a description of the meaning of a change of colour of the ink layer or provide any additional information.

According to one embodiment, the protective layer is transparent. Thus, any text or information printed on the semi-permeable layer will be visible to the end consumer since the protective layer is arranged above the semi-permeable layer. The semi-permeable layer is the layer arranged the furthest away from the barrier film top lidding when the food quality indicator is arranged in a food package.

According to one embodiment, the ink layer and the semi-permeable layer have different surface areas so the ink layer and the semi-permeable layer can be fastened to the second side of the double-sided adhesive layer.

According to one embodiment, the first side of the adhesive layer of the food quality indicator is configured to be attached to a barrier film of a food package. Thus, the food quality indicator is attached to a food package in an easy manner.

According to one embodiment, the food quality indicator is configured to be attached inside a food package containing food products producing analytes upon decomposition, such as meat, poultry, fish, dairy products, fruits vegetables, processed food.

According to one embodiment, the ink is made of a surfactant, a thickening agent, a pigment indicator, a benzopyrone compound, a solvent and a colour enhancer.

According to one embodiment, the surfactant constitutes between 10% to 30% of the ink.

According to one embodiment, the surfactant is selected from a group consisting of polyelectrolytes, hydrocolloids or any combinations thereof.

According to one embodiment, the thickening agent constitutes between 30% to 50% of the ink.

According to one embodiment, the thickening agent is a polymer thickening agent.

According to one embodiment, the thickening agent is selected from the group consisting of cellulose polymers, carbomer polymers, a carbomer derivative, a cellulose derivative, agar-agar, polyacrylamide, polyvinyl alcohol, alginic acid, poloxamers, polysaccharides, gelatin, pectin or any combinations thereof.

According to one embodiment, the pigment indicator is coupled to alkoxysilane.

According to one embodiment, the alkoxysilane coupled to the pigment indicator constitutes between 0.1% to 10% of the ink.

According to one embodiment, the pigment indicator is an organic pigment selected from the group consisting of diketopyrrolopyrroles, quinophthalones, azo pigments, zingiberaceae, quinacridones, indanthrones, flavanthrones, pyranthrones, anthraquinones, perylenes, dioxazines, perinones, thioindigo, isoindolines, and isoindolinones or any combinations thereof.

According to one embodiment, the alkoxysilane compound is selected from the group consisting of a non-amino silane compound such as (3-acryloxypropyl) trimethoxysilane, 3-mercaptopropyl triethoxysilane, 3-mercaptopropyl trimethoxysilane, methacryloxyloxy propyl trimethoxysilane or any combinations thereof.

According to one embodiment, the ink is further made of benzopyrone compound such as Coumarin.

According to one embodiment, the solvent is selected from polyester resins, phenolic resins, alkyd resins, polycarbonate resin, polyamide resins, polyurethane resins, silicon resins, epoxy resin, polyethylene resins, acrylic resins, polystyrene resins, polypropylene resins or any combinations thereof.

According to one embodiment, the colour enhancer constitutes between 0.5% to 1% of the pigment indicator.

According to one embodiment, the colour enhancer is a phthalocyanine.

According to one embodiment, the porous substrate of the ink layer is selected from cellulose, paper, synthetic paper, polyolefin-based synthetic material, thermoplastic copolyester (TPPE), low-density or high-density polyethylene, low-density or high-density polypropylene, nylon, nonwoven fabric, fabric or polymeric membranes.

According to one embodiment, the porous substrate of the ink layer has pores with pore size between 10 μm to 100 μm, with a thickness ranging between 0.05 millimetre to 0.5 millimetre, preferably between 0.05 milometer to 0.1 millimeter.

According to a second aspect, a method for preparing an ink of a food quality indicator is provided. The method includes preparing a mixture by dissolving a surfactant in a solvent and adding pigment coupled to silane to the mixture. Then, the mixture is treated with ultrasonicating for 10-30 minutes followed by vortex mixing for 20-30 minutes. A colour enhancer and a polymer thickening agent are added to the mixture. The mixture is thereafter treated by ultrasonicating for 10-30 minutes followed by vortex mixing for 20-30 minutes. Subsequently, a coupling agent is added to the mixture and the mixture is heated to 30-60° C.

An advantage of the ink of the food quality indicator being provided according to the present method is that the ink changes colour in the presence of analytes such as volatile organic compounds (VOCs), volatile gases, volatile amines, biogenic amines, or volatile sulphur compounds. Thus, the ink can be arranged in a food quality indicator described above that has several layers for protecting the ink and thus enabling a controlled migration of analytes to the ink when the ink is arranged inside a food package.

According to a third aspect, a method for preparing a protective layer for a food quality indicator is provided. The method includes the steps of providing a polymer and a solvent at a weight ratio of 0.01:1, preferably 0.15:0.56 and then dissolving the polymer in the solvent at room temperature.

An advantage of the protective layer provided according to the present method is that it is hydrophobic and permeable to gases. Most substrates are highly porous substrates which are permeable to both liquids and gases. Achieving a selective diffusion on permeable and highly permeable substrates is a challenge in particular when it comes to differentiating between gases and liquids which is overcome by the protective layer of the present invention. The polymer of the protective layer is selected from a group consisting of polydimethylsiloxane (PDMS), polyurethane, polyamide or polyethylene, polyvinyl alcohol (PVA), polyvinyl chloride (PVC), ethyl cellulose (EC), methyl cellulose (MC), hydroxypropyl methylcellulose (HPMC), or any combinations thereof with a weight ratio between 0.1 to 0.5. The solvent of the protective layer is selected from a group of solvents having a viscosity between 20 to 300 cP and a boiling point between 34.6° C. to 190° C., more preferably between 50 to 90° C. The solvents have vapour pressures between 2.9 kPa to 170.6 kPa, preferably between 21.6 kPa to 110.6 kPa

According to a fourth aspect, a method for manufacturing an indicator of the food quality indicator is provided. The method includes the step of providing an ink according to the above mentioned method for preparing the ink. Followed by applying a layer of the ink to a porous substrate to provide an ink layer. The first side of the ink layer is attached to a semi-permeable layer. The second side of the ink layer is attached to a second side of a double-sided adhesive.

An advantage of the present method for manufacturing an indicator of the food quality indicator is that the food quality indicator comprises several layers to protect and enclose the ink inside the food quality indicator in order to provide a controlled migration of analytes such as volatile organic compounds (VOCs), volatile gases, volatile amines, biogenic amines, or volatile sulphur compounds to the ink. Thus, the change of colour of the ink is not random. The ink is also kept from being smudged out and thus not changing colour as efficiently upon entering in contact with analytes. The double-sided adhesive layer enables the food quality indicator to be securely fastened to a food package in an easy manner.

According to one embodiment, the method further includes the step of providing a protective layer according to the method for preparing the protective layer mentioned above and coating the semi-permeable layer with the protective layer prior to applying the ink layer to the semi-permeable layer so that the protective layer is arranged between the semi-permeable layer and the ink layer. The steps of the method relating to the protective layer are only to be included in the method if the food quality indicator is to be provided with a protective layer.

According to one embodiment, the last step of the method is to fasten the first side of the double-sided adhesive to a barrier film for a food package.

### BRIEF DESCRIPTION OF THE DRAWINGS

These and other aspects, features and advantages of which the invention is capable, will be apparent and elucidated from the following description of embodiments of the present invention, reference being made to the accompanying drawings, in which:

FIG. 1 is a perspective view of the food quality indicator on a food package as an end user would see the indicator on the package;

FIG. 2A-2E are front views of the food quality indicator illustrating a progression of the food quality indicator, where FIG. 2A shows a food quality indicator that has not yet detected any analytes in the food package and FIG. 2E shows a food quality indicator showing that the food inside the food package is no longer edible;

FIG. 3A is an exploded perspective view illustrating a number of layers comprised in a food quality indicator according to a first embodiment of the invention;

FIG. 3B is a cross-sectional view of the food quality indicator illustrating said layers comprised in the food quality indicator according to the first embodiment to the invention;

FIG. 4A is an exploded perspective view illustrating a number of layers comprised in a food quality indicator according to a second embodiment of the invention;

FIG. 4B is a cross-sectional view of the food quality indicator illustrating the layers comprised in the food quality indicator according to the second embodiment of the invention;

FIG. 5 is a flow chart illustrating a method of preparing the ink that changes colour in the presence of analytes, volatile gases and/or biogenic gases according to the first or second embodiment;

FIG. 6 is a flow chart illustrating a method of preparing a protective layer for the food quality indicator according to a second embodiment; and

FIG. 7 is a flow chart illustrating a method of preparing the food quality indicator according to the first or second embodiment.

### DESCRIPTION OF THE INVENTION

Embodiments of the presentation invention will be described in more detail below with reference to the accompanying drawings in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the detailed description of the particular embodiments illustrated in the accompanying drawings is not intended to be limiting of the invention.

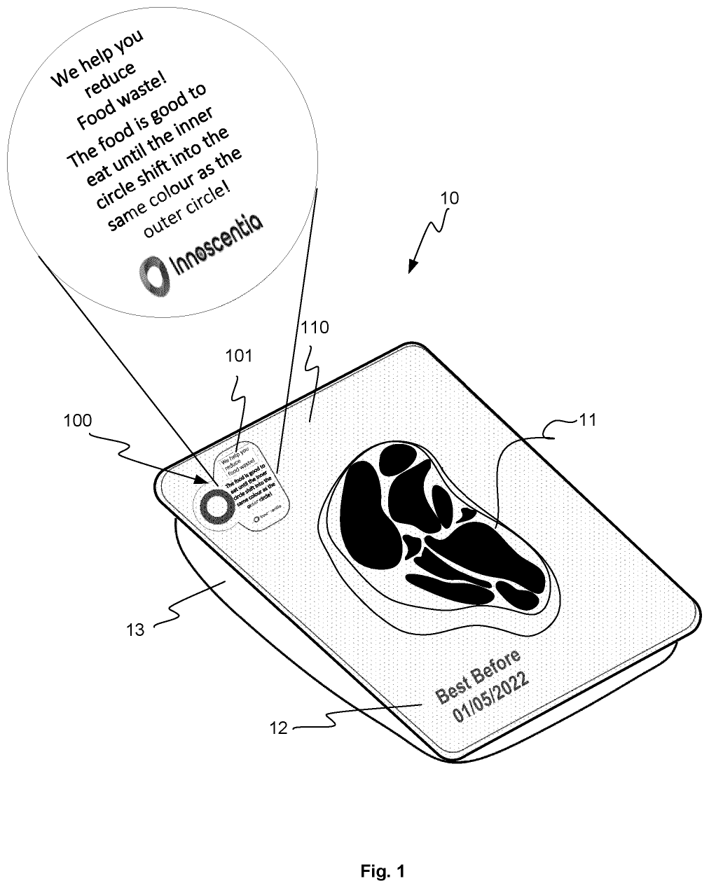

Referring to FIG. 1, a food product 11, namely a piece of meat is shown enclosed in a food package 10. The food package 10 is a food tray 13 with a top lidding barrier film 110.

As in known, such a food package 10 comprises a “best before date” or “use by date” to indicate of the lifespan of the food product. In FIG. 1, a “best before date” 12 is provided on the barrier film 110 of the food package 10.

The food product 11 is illustrated as a piece of meat as an example. Instead of a piece of meat, poultry, fish, dairy products, fruits vegetables, processed food or any other type of perishable food could have shown in the food package 10 of FIG. 1.

FIG. 1 further describes a food quality indicator 100 according to the present invention. The food quality indicator 100 has a reactive ink 131 absorbed on a porous substrate 132 that changes colour as a function of quality the of the food being monitored. Thus, providing means to determine whether a food product 11 is edible or not.

The indicator 100 may be provided as a label, pouch, sachet, hollow tube, sheet or tea-bag. The colour changing material is provided between two substrates.

The indicator 100 should be arranged in the same space as the food being monitored by the indicator 100. In FIG. 1, the indicator 100 is thus attached to the barrier film 110 and is located within the area formed by the barrier film 110 and the food tray 13.

When packaging the food product 11, the food product 11 is arranged inside the food tray 13. The barrier film 110, with the food quality indicator 100 attached to the barrier film 100, is arranged to cover the food tray 13 and provide a top lidding to the food package 10.

The barrier film, to which the food quality indicator 100 is attached, may be manufactured from a composition comprising of at least one of:
polyolefins, which include low-density (LDPE), linear low-density polyethylene (LLDPE), and high-density polyethylene (HDPE), polypropylene (PP), and biaxially-oriented polypropylene (BOPP);
copolymers of ethylene, like ethylene-vinyl acetate (EVA), ethylene-vinyl alcohol (EVOH), and ethylene-acrylic acid (EAA);
composites like amorphous silicon oxide (SiOx) and other ceramics coated polymer;
nanocomposite polymers like polymer reinforced nanoparticles;
substituted olefins, like polystyrene (PS), high-impact polystyrene (HIPS, with 1,3-butadiene isomer added during the polymerization of the PS), oriented polystyrene (OPS), poly(vinyl alcohol) (PVOH), poly(vinyl chloride) (PVC), and poly(vinylidene chloride) (PVdC), and poly(tetrafluoroethylene) (PTFE);
polyesters, like polyethylene terephthalate (PET), polyethylene naphthalate (PEN), and relative copolymer PET-PEN;
polycarbonates (PC);
polyamide (PA);
acrylonitriles, like polyacrylonitrile (PAN) and acrylonitrile/styrene (ANS);
regenerated cellulose;
polylactic acid (PLA) as biodegradable polymer for food packaging contact;
plasma pre-treated polymers.

The barrier film thickness is between 10 μm to 10,000 μm, preferably between 100 μm to 1000 μm.

The barrier film is a transparent film with a luminous transmittance and haze range between 0% to 95% and 0% to 5% respectively, preferably between 50% to 90% and 1% to 3% respectively.

In the simplest form, the indicator 100 comprises the above-mentioned reactive ink 131, also called ink layer 130 as the ink 131 is absorbed on a porous substrate 132 provided as a layer of the indicator 100. The ink layer 130 is sandwiched between two substrates, and has a function to undergo a systematic colour change in the presence of analytes such as volatile organic compounds (VOCs), volatile gases or biogenic gases.

The colour change of the reactive ink 131 is substantially proportional to the amount of detected analytes such as VOCs, volatile gases or biogenic gases. The reactive ink 131 can detect the most common analytes including Alcohols, Amines, Aldehydes, Ketones, Esters and Sulphides, volatile amines including but not limited to methylamine, and ethyl amines, volatile gases such as volatile fatty acids, biogenic amines and volatile sulphur compounds.

As mentioned above, the ink layer 130 is made by absorbing reactive ink 131 on a porous substrate 132. The porous substrate 132 has a function of absorbing water-based or solvent based reactive ink 131 and preventing the migration of the reactive ink 131 into a food product. Porous substrates are commercially available and physically changed the dimensions as per the size of the label 101. The porous substrates are typically made from cellulose, paper, synthetic paper, polyolefin-based synthetic material, thermoplastic copolyester (TPPE), low-density or high-density polyethylene, low-density or high-density polypropylene, nylon, nonwoven fabric, fabric or polymeric membranes to name a few, containing pore sizes between 5 μm to 100 μm.

In one embodiment of the present invention, the thickness of the ink layer 130 is typically between 0.05 millimeter to 0.5 millimeter, preferably between 0.05 to 0.1 millimeter.

The advantageous feature of the ink layer 130 with the use of porous substrate 132 is strongly hinder the migration of ink as well as provides high background contrast so that any minor changes in the colour due to the ink reaction with analytes is visible and clear. In addition, the ink layer 130 also provides case of integration between the protective layer 150 and adhesive layer 120, hence production process is simplified and scaled.

As is illustrated by FIG. 1, the indicator 100 is a label 101 attached to the transparent barrier film 110 so that the user can see and read the indicator 100 through the barrier film 110 of the food package 10.

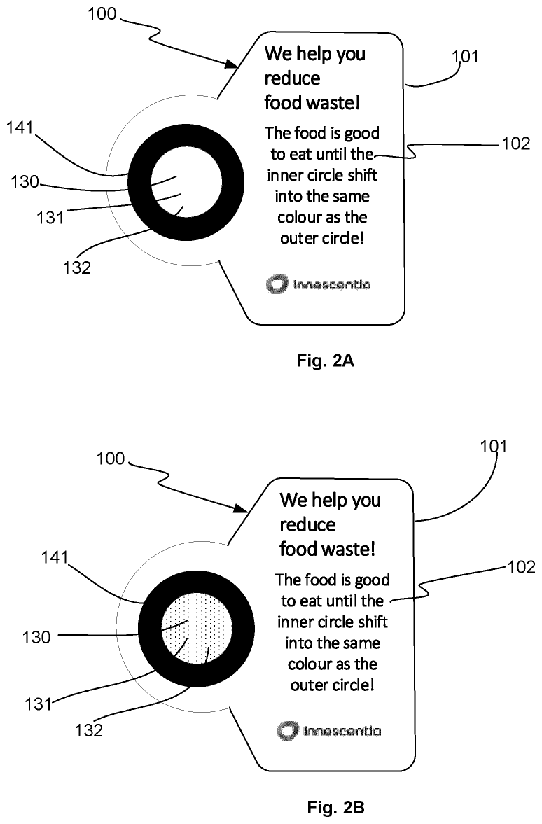

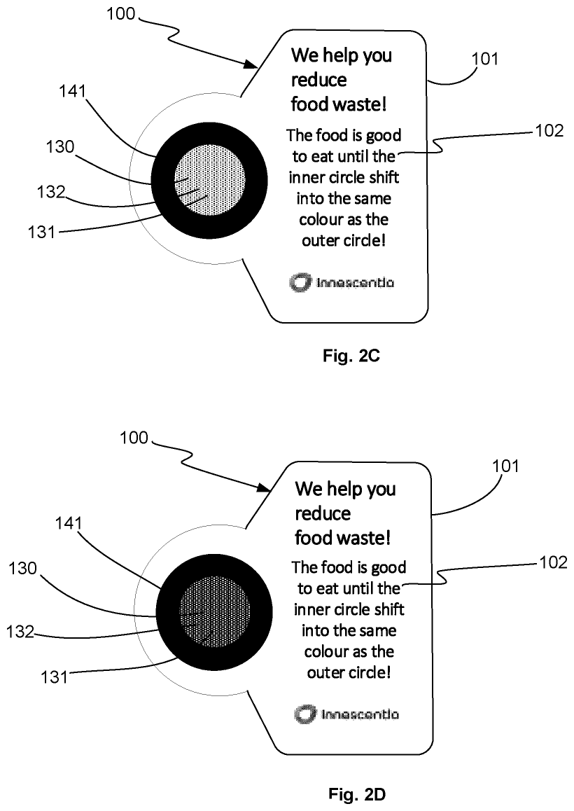

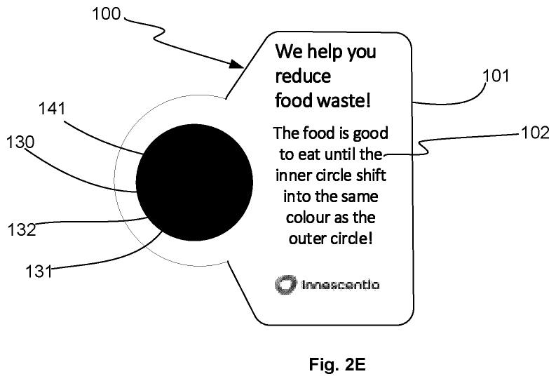

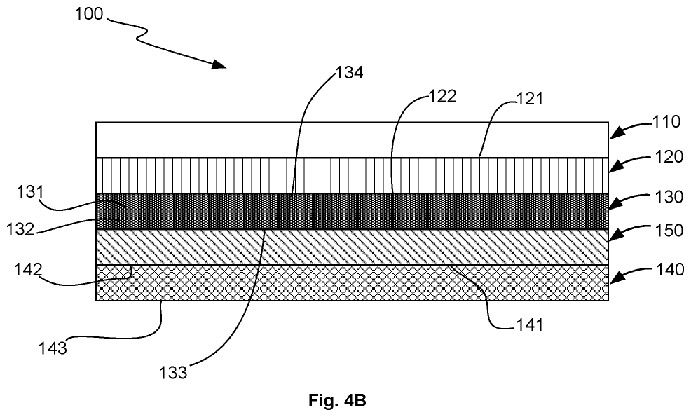

FIGS. 2A-2E illustrate a progression of a food quality indicator 100 as perceived by an end customer. The indicator 100, as is shown in FIGS. 2A-2E, comprises the ink layer 130 and a reference area 141. The reference area 141 is preferably part of a different layer of the indicator 100 than the ink layer 130 as will be disclosed with reference to FIGS. 4A-B and 5A-B.

The indicator 100 may further comprise instructions 102 for interpreting the ink layer 130 in relation to the reference area 141. By way of example, the ink layer 130 in FIGS. 2A-2E is shown as a circular area encircled by the reference area 141. The reference area 141 and the ink layer 130 should be placed in proximity to each other so that it is easy to compare the colours of the two areas.

As previously mentioned, the indicator 100 may further comprise instructions 102 for interpreting the colour of the ink layer 130. Such instructions 102 could be provided as printed text such as in FIGS. 2A-2E, bar codes, additional reference areas, “an expiry date” or “use before date” or any other relevant indication or information that may help the user decide whether the food product 11 in the food package 10 is edible.

FIG. 2A shows a food indicator 100 according to the present invention prior use and/or that has not yet detected any analytes, volatile gases or biogenic gases. Therefore, the reactive ink 131 of the ink layer 130 is white in this example whereas the reference area 141 is shown as black. The contrast difference between the ink layer 130 and the reference area 141 is high.

In FIG. 2B, the food indicator 100 has detected some analytes, volatile gases or biogenic gases. Still, the detected amount is low and therefore the food is deemed to be edible. This is shown by some dots and/or small areas of the ink layer 130 being coloured in a darker tone which resembles the colour of the reference area 141. The contrast difference between the ink layer 130 and the reference area 141 is still clear. Thus, the food is comestible.

In FIG. 2C, the food indicator 100 has detected some additional analytes, volatile gases or biogenic gases compared to what was shown in FIG. 2B. This is shown by additional dots and/or small areas of the ink layer 130 being coloured in a darker tone similar to the colour of the darker tone. There is now less visible contrast between the ink layer 130 and the reference area 141. Thus, the food is edible but will soon be spoiled.

In FIG. 2D, the food indicator 100 has detected additional analytes, volatile gases or biogenic gases. There is almost no contrast left between the ink layer 130 and the reference area 141. The food is most certainly to comestible anymore.

In FIG. 2E, the food indicator 100 has detected additional analytes, volatile gases or biogenic gases. There is no contrast between the ink layer 130 and the reference area 141. The food in the monitored food package is not comestible.

In FIGS. 2A-2E, the colour changes have been illustrated with the reference area 141 being black and the ink layer 130 being white. The invention is not limited to these colours and this arrangement. Depending on the ink 131 and depending on the concentration of analytes released by perishable products, the colour of the indicator 100 could for instance changes from white to blue and finally to brown, yellow to magenta, orange to magenta to name a few colour changes.

The reference area 141 is adapted to fit the ink layer 130 comprising ink 131 absorbed on a porous substrate 132. The reference area 141 can be sectioned into several reference areas with different colour so that it is possible to follow the development of the food product 11 until it has perished. For instance, the ink layer 130 being white could for instance mean that the food is fresh and no analytes have yet been detected. A colour change from white to blue could indicate that the food is still fresh but some analytes have been detected. Thus, it is possible to get an indication of the state of the food until it has perished. A change of colour from orange to magenta would then, as an example, mean that the food product 11 is perished. The reference area 141 could then also be provided with a text area to provide a description of the meaning of a change of colour for the ink layer 130.

It is also possible to provide means for wireless communication on the indicator 100 so that the user is notified of the status of the product being monitored by the indicator 100. Thus, a perishing product can be identified at an early stage and necessary precautionary measures can be taken.

The ink 131 according to the invention is a composite of a food grade surfactant, a food grade thickening agent, a pigment indicator, a food grade benzopyrone compound, a solvent and a colour enhancer.

The reactive ink 131 of the ink layer 130 more specifically comprises:
a. the food grade polymer surfactant that constitutes between 10-30% of the total ink. The food grade surfactant is selected from a group consisting of polyelectrolytes, hydrocolloids or any combinations thereof;
b. the food grade thickening agent that constitute 30-50% of the total ink. The food grade thickening agent is a polymer thickening agent. The thickening agent is selected from the group consisting of cellulose polymers, carbomer polymers, a carbomer derivative, a cellulose derivative, agar-agar, polyacrylamide, polyvinyl alcohol, alginic acid, poloxamers, polysaccharides, gelatin, pectin or any combinations thereof;
c. Alkoxysilane coupled to the pigment indicator that constitutes 0.1-10% of the total ink.

The pigment indicator is an organic pigment selected from the group consisting of diketopyrrolopyrroles, quinophthalones, azo pigments, Zingiberaceae, quinacridones, indanthrones, flavanthrones, pyranthrones, anthraquinones, perylenes, dioxazines, perinones, thioindigo, isoindolines, and isoindolinones or any combinations thereof.

The alkoxysilane compound may be selected from the group consisting of a silanes without amino groups such as (3-acryloxypropyl) trimethoxysilane, 3-mercaptopropyl triethoxysilane, 3-mercaptopropyl trimethoxysilane, methacryloxyloxy propyltrimethoxysilane or any combinations thereof.

The silane coupled pigment indicator is prepared by dissolving 0.1% to 15% of pigment in alkoxysilane solvent and reflux the solution under 40 to 60° C. for approximately 3 to 10 hours;
d. the food grade benzopyrone compound such as Coumarin.
e. the solvent dissolves the components of a, b, c, and d that constitutes 40-75% of the total ink. The solvent is provided for mixing the components of the ink 131.

The clear solvent is chosen from polyester resins, phenolic resins, alkyd resins, polycarbonate resin, polyamide resins, polyurethane resins, silicon resins, epoxy resin, polyethylene resins, acrylic resins, polystyrene resins, polypropylene resins, singly or in mixtures, which is 0.5-10% of the solvent used.
f. A colour enhancer, that constitutes 0.5-1% of the pigment indicator. The color enhancer is a phthalocyanine.

The ink has the function of undergoing a systematic colour change upon binding to or reacting with analytes, volatile organic compounds, volatile gases and/or biogenic gases.

The porous substrate 132 of the ink layer 130 more specifically comprises a porous substrate selected from commercially available porous substrates with pore size between 10 μm to 100 μm, with a thickness ranging between 0.05 millimetre to 0.5 millimetre, preferably between 0.05 milometer to 0.1 millimeter.

The porous substrate 132 of the ink layer 130 more specifically selected from the group of commercially available porous substrates such as cellulose, paper, synthetic paper, polyolefin-based synthetic material, thermoplastic copolyester (TPPE), low-density or high-density polyethylene, low-density or high-density polypropylene, nylon, nonwoven fabric, fabric or polymeric membranes.

The ink 131 on the porous substrate 132 of the ink layer 130 is prepared by 1) drop casing: the ink 131 is dropped on a porous substrate with a volume between 1 μl to 10 μl, preferably between 3 μl and 6 μl, and dried at ambient conditions, or by 2) dip coating: the porous substrate 132 is dipped into the ink 131 solution for 10 sec and dry under ambient conditions.

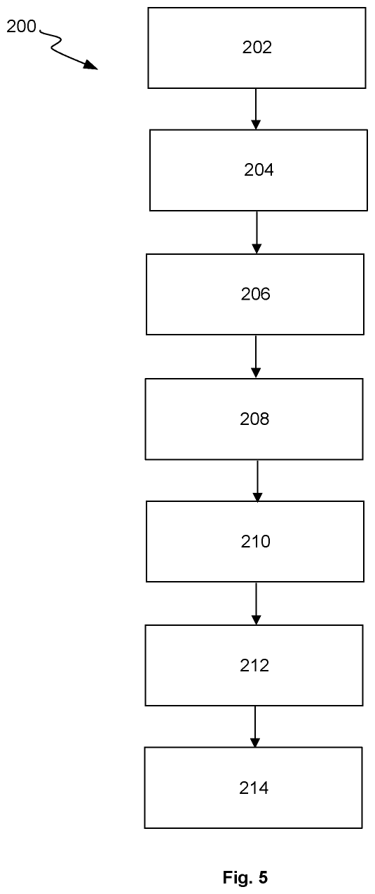

FIG. 5 comprises a flow chart illustrating a method 200 for preparing the reactive ink 131 that changes colour in the presence of analytes, volatile gases and/or biogenic gases. A detailed description of the compounds of the reactive ink 131 of the ink layer 130 are provided above. Preparations include providing the pigment and the alkoxysilane according to the specifications above.

The alkoxysilane coupled pigment indicator is prepared by dissolving 0.1% to 15% of pigment in alkoxysilane solvent and refluxing the solution under 40 to 60° C. for 3 to 10 hours. The pigments are treated with functionalized silanes to improve adhesion properties.

At least one of the food grade polymer surfactant, the solvent, the colour enhancer, the polymer thickening agent and the food grade benzopyrone are provided according to the specifications provided above.

The method 200 for preparing the reactive ink 131 comprises the steps of preparing 202 a mixture by dissolving the food grade polymer surfactant in the solvent.

Then, the pigment coupled with alkoxysilane is added 204 to the mixture.

Subsequently, the mixture is treated 206 by ultrasonication for 10-30 minutes followed by vortex mixing for 20-30 minutes.

The colour enhancer and the polymer thickening agent are added 208 to the mixture.

Thereafter, the mixture is again treated 210 by ultrasonication for 10-30 minutes followed by vortex mixing for 20-30 minutes.

Subsequently, the coupling agent is added 212 to the mixture and the mixture is heated 214 to 30-60° C.

In a first embodiment, the ink 131 is sandwiched between two substrates forming the active ink layer 130 changing colour in the presence of analytes such as volatile organic compounds (VOCs), volatile gases and/or biogenic gases.

In the simplest form, one of the two substrates is an adhesive layer 120 and the other one is a semipermeable layer 140, permeable to gases and impermeable to liquids.

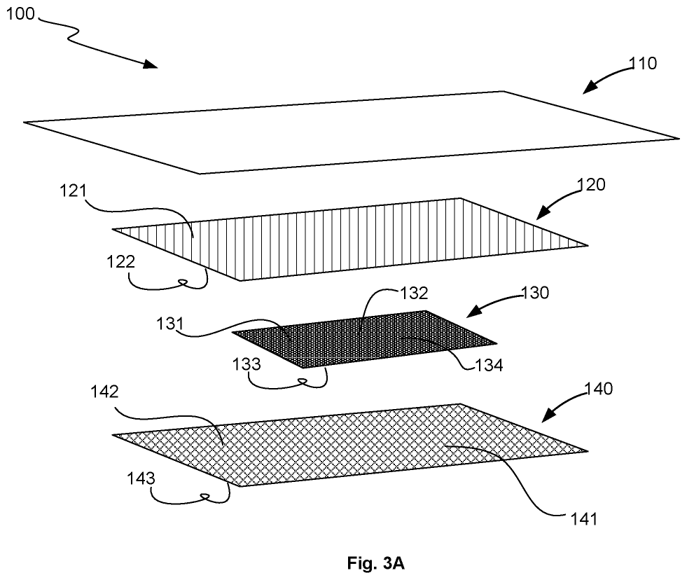

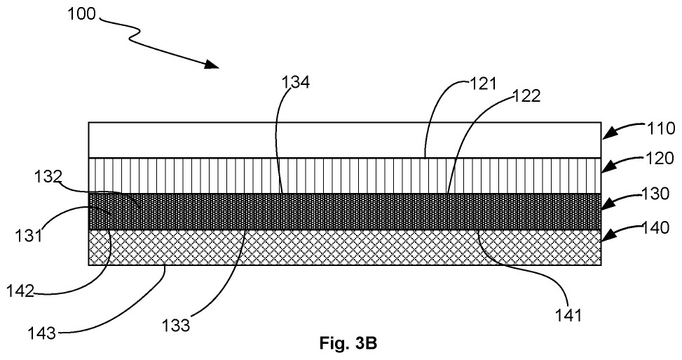

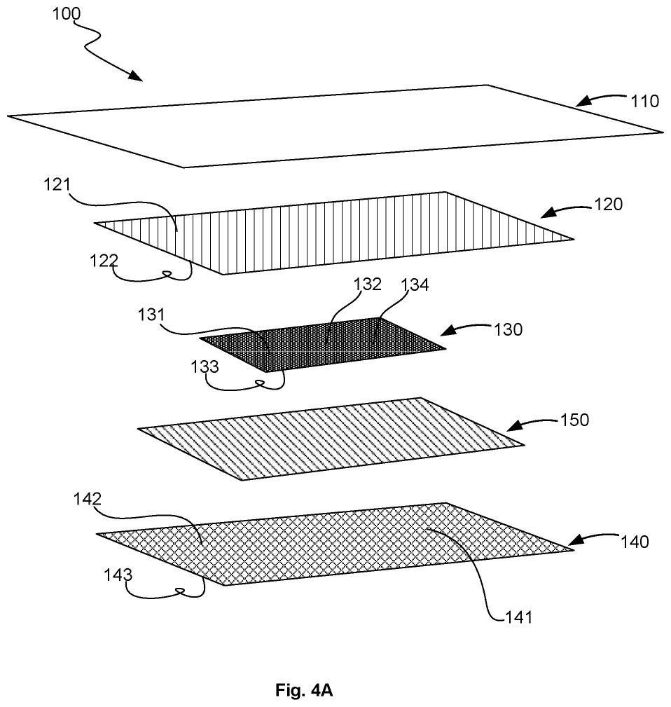

Thus, the indicator 100 has an adhesive layer 120, an ink layer 130, and a semipermeable layer 140. The ink layer 130 is arranged between the adhesive layer 120 and the semipermeable layer 140. This is illustrated in FIG. 3A and FIG. 3B. The semipermeable layer 140 has a first surface 142 and a second surface 143. The adhesive layer 120 is a double-sided adhesive layer and is the only fastener needed to fasten the layers of the indicator 100. Thus, providing the indicator 100 as a single piece to be secure to a barrier film 110. The indicator 100 is attached to the barrier film by the adhesive layer 120 as well.

FIG. 3A shows an exploded perspective view of the layers comprised in the food indicator 100 according to the first embodiment of the invention. The width and the length of the ink layer 130 are lesser than the width and the length of the double-sided adhesive layer 120 and the semipermeable layer 140 as is illustrated in FIG. 3A. Thus, both the ink layer 130 and the semipermeable layer 140 are in direct contact with the adhesive layer 120 and are fixedly attached so the indicator 100 is provided in as a single unit.

FIG. 3B is a cross-sectional view of the food quality indicator 100 illustrating the layers comprised in the food quality indicator 100 according to the first embodiment to the invention. The surface area differences between the ink layer 130, the adhesive layer 120 and the semipermeable layer 140 are omitted from FIG. 3B. The purpose of FIG. 3B is to provides an illustration of how the layers of the indicator 100 are arranged upon each other.

An additional layer is shown in FIGS. 3A and 3B, namely a top plastic layer 110. When the indicator 100 reaches the end customer, the indicator 100 is attached to the top plastic layer 110 such as the barrier film 110 described above. The indicator 100 is attached in such way that it is able to detect the spoilage of the food contained in the food wrap or container. The indicator 100 is preferably fastened to the inside of the food wrap or container, such as the barrier film 110 shown in FIG. 1, so that the food and the indicator 100 are comprised in the same compartment. Thus, the indicator 100 is exposed to the food product and can measure emitted analytes, volatile gases or biogenic gases.

The analytes migrate through the semi-permeable layer 140 to reach the ink layer 130. The semi-permeable layer 140 is preferably arranged facing the food product 11 being monitored. The adhesive layer 120, attached to the barrier film, is thus placed the furthest away from the food product 11. The adhesive layer 120 is impermeable to gases.

The adhesive layer 120 of the indicator 100 comprises a first surface 121 and a second surface 122, wherein the first surface 121 of the adhesive layer 120 is fixated to the top plastic layer 110 and the second surface 122 of the adhesive layer 120 is fixated the reactive ink layer 130 and to the semipermeable layer 140. The ink layer 130 is provided between the adhesive layer and the semipermeable layer 140.

Prior use, the first surface 121 of the adhesive layer 120 is covered by a liner that is removed before attaching the adhesive layer 120 to the plastic layer 110. Thus, the function of the adhesive layer 120 is to fixate the indicator 100 to the top plastic layer 110 and but also to fixate all layers of the indicator 100 together.

The adhesive used shall be approved for indirect contact with all types of food. The adhesive layer is transparent.

The double-sided adhesive substrate 120 requires high adhesion strength between the tape and the surface of a substrate, but also requires high transparency between layers of the tape. Double-sided adhesives are available commercially and are tackified polyurethane foam, Acrylic adhesive, rubber adhesive, and typically backing by a polyester (PET) film or a PVC film.

The double-sided adhesive used is preferably AM413, TT BG40WH S445N-BG40BR by Avery Dennison. The adhesive is particularly suitable for rough or difficult substrates such a rubber goods, packaging materials such as plastic container. The adhesive has suitable performance at low temperature, minimum application temperature is −5° C. and service temperature is −40° C. to 70° C.

Avery Dennison adhesive S445N has been tested by a certified laboratory and has received a certificate of compliance. S445N complies with European food regulation 1935/2004/EC, with the German Recommendation (BfR) XXI and with FDA § 175.105. It also meets the demands of the limit values laid down in 10/2011/EU. It further complies with DIN ISO 3826-1.

In accordance with the requirements of relevant EU food regulations, the adhesive S445N may safely stand in direct contact with dry, (moist) and non-fatty foodstuffs. Furthermore, the seams (edges) of the label material may safely come into direct contact with all kinds of foodstuffs.

The ratio of food contact surface area to volume when considering labels on a package is 6 dm²/kg. The ratio of food contact surface area to volume when considering direct labelling on foodstuffs is 2 dm²/kg.

According to FDA regulations, the adhesive may come into direct contact with dry foodstuffs, or alternatively it must be separated from the foodstuffs by a functional barrier.

The semipermeable layer 140 of the indicator 100 is permeable to gases only and impermeable to liquids.

Preferably, the substrate used for the semipermeable layer 140 that is permeable to gases is a woven or non-woven fibrous sheet material made of polyolefin, polyethylene fibres, polypropylene, polyester, or plant fibres, Polylactic acid (PLA), polyvinylidene difluoride (PVDF), permeable Teflon such as stretched polytetrafluoroethylene (PTFE), fluoroplastic, polycarbonate, cellulose (regenerated cellulose, cellulose acetate, cellulose nitrate and cellulose ester), polyethersulfone (PES), nylon, or a combination of them and are commercially available.

The semipermeable layer 140 provides a base for the indicator 100 and is the substrate arranged the furthest away from the barrier film. The semipermeable layer is arranged toward the food product being monitored when the indicator 100, provided as a label, is arranged in a food package such as the food package 10 of FIG. 1.

An essential feature of the semipermeable layer 140 is that it is permeable to gases so that analytes such as volatile organic compounds, volatile gases and/or biogenic gases located produced by the monitored food upon decomposition can move/migrate across the semipermeable layer 140 and reach the ink layer 130. Thus, the ink 130 will react with the volatile organic compounds (VOCs), volatile gases and/or biogenic gases and result in a change of colour for the ink 130.

The semipermeable layer 140 is non-transparent and has a function of providing a surface for printing the reference area 141 and/or any further text, instructions for interpreting the colours of the ink layer 130, bar codes, additional reference areas and any other relevant indication or information. The semipermeable layer being non-transparent augments the visibility of the information printed thereon. The semipermeable layer 140 allows for gases to pass but hinders migration of ink.

A particularly preferred substrate for the semipermeable layer 140 is a non-woven, spunbonded polyester or polyolefin, such as the substrate from under the tradename TYVEK® from DuPont. In the present invention, TYVEK® 1059B and 1073B spun-bonded polyolefin (non-woven high-density polyethylene fibres) are preferred because these materials are approved by the FDA, USDA and EU REACH for direct food contact.

The semipermeable layer 140 which is permeable to gases and impermeable to liquids may also be a paper, a synthetic paper, a thermoplastic copolyester (TPPE), polyolefin-based synthetic material, whose permeability depends on the particle retention, where the particle retention is below 5 μm.

A second embodiment of the present invention will be described with FIGS. 4A and 4B. FIG. 4A shows an exploded perspective view illustrating the layers comprised in the food indicator 100 according to the second embodiment of the present invention.

In a second embodiment, the ink 130 is sandwiched between the adhesive layer 120, same adhesive as described for the first embodiment, and the semipermeable layer 140, same semipermeable layer as described for the first embodiment. All features described in the first embodiment are also present is the second embodiment. The reference signs correspond.

The indicator 100 according to the second embodiment also comprises a protective layer 150. The protective layer 150 is arranged between the ink layer 130 and the semipermeable layer 140. The protective layer 150 is preferably coated on the semipermeable layer 140.

The dimensions described for the adhesive layer 120, the ink layer 130 and the semipermeable layer 140 applies to the second embodiment. Everything described for the first embodiment applies to the second embodiment as well.

FIG. 4A shows that the width and the length of protective layer 150 is superior to the corresponding dimensions for the ink layer 130 and inferior to the corresponding dimensions for the semipermeable layer 140. Thus, the ink layer 130, protective layer 150 and the semipermeable layer 140 are in direct contact with the second surface 122 of the adhesive layer 120 and are fixedly attached so the indicator 100 is provided in as a single unit.

FIG. 4B illustrates a cross-sectional view of the food indicator 100 according to the second embodiment to the invention. The layer dimensions are omitted from FIG. 4B and all layers of the indicator 100 are shown having the same width and length. The purpose of FIG. 4B is to provides an illustration of how the layers of the indicator 100 are arranged upon each other.

An additional layer is shown in FIGS. 4A and 4B, namely the top plastic layer 110. As has been described above, when the indicator 100 reaches the end customer, the indicator 100 is fixed to the top plastic layer 110 such as the barrier film 110.

The purpose of having a protective layer 150 is to hinder migration of ink both from the ink layer 130 and the semipermeable layer 140. The protective layer 150 is permeable to gases and hence works as a functional barrier.

The protective layer 150 only permits gases to diffuse through it. The analytes migrate through the semi-permeable layer 140 and the protective layer 150 to reach the ink layer 130. Ink and liquids are prevented from diffusing across the material of the protective layer 150.

The protective layer 150 is a chemically and mechanically stable hydrophobic layer and works as a functional barrier. The protective layer 150 is made of a composite comprising of a polymer and a solvent.

The polymer is one of the materials selected from the Polydimethylsiloxane (PDMS), polyurethane, polyamide, polyethylene, Polyvinyl alcohol (PVA), polyvinyl chloride (PVC), ethyl cellulose (EC), methyl cellulose (MC), hydroxypropyl methylcellulose (HPMC), and combinations of them with a weight ratio between 0.1 to 0.5.

The solvent is selected from a range of solvents having a viscosity between 20 to 300 cP. Preferably, the selected solvent has a boiling point between 34.6° C. to 190° C., preferably between 50 to 90° C. In addition, the solvent is also selected from a range of solvents having vapour pressures between 2.9 kPa to 170.6 kPa, preferably between 21.6 kPa to 110.6 kPa. Thus, quick drying is achieved.

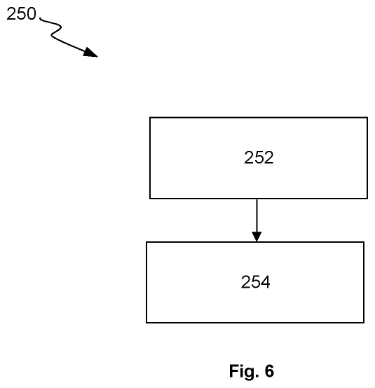

FIG. 6 is a flow chart illustrating a method 250 for preparing the protective layer 150. The method comprises the steps of providing 252 the polymer and the solvent at a weight ratio in the interval 0.01:2 to 0.2:0.4, such as about 0.01:1, preferably about 0.15:0.56 and dissolving 254 the polymer in the solvent at room temperature.

After making protective layer 150, the protective layer 150 is coated on the semipermeable layer 140. The protective layer 150 is coated on the semipermeable layer 140 using standard printing methods such as screen print, flexographic print, digital print, and Gravure print. Once, the coating is done, the active substrate is quickly dried in a heating element at 80° C. for 1 second to 5 minutes, particularly between 3 to 30 seconds. This will be described further below.

The protective layer 150 may or may not be transparent. If the solvent 150 is transparent, information may be printed on the semipermeable layer 140.

The additional protective layer 150 makes the indicator 100 safer since ink from the ink layer 130 is prevented from migrating across protective layer 150. Thus, providing an additional barrier between the ink and the food being monitored.

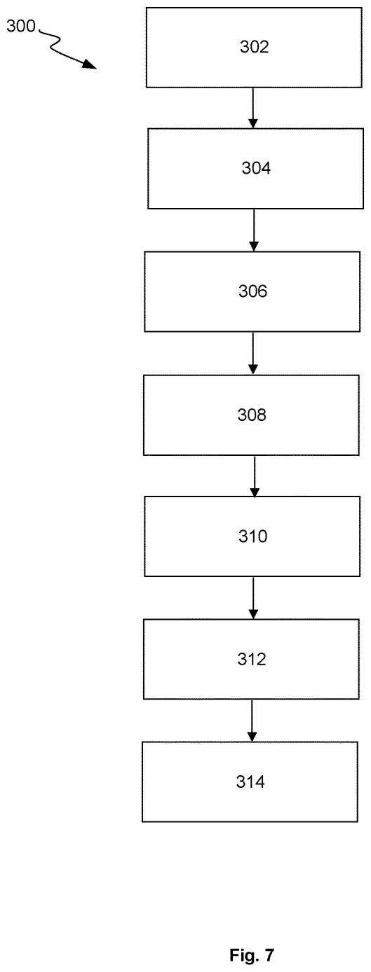

FIG. 7 is a flow chart illustrating a method 300 for making the indicator 100 according to the first or the second embodiment of the present invention. A detailed description of the different layers of the indicator 100 are provided above.

A reference area 141 or a text can be printed on the semi-permeable layer 140.

The method 300 comprises the steps of providing 306 an ink according to the method 200 for preparing the ink 131. The ink 131 is applied 308 on, and absorbed by, the porous substrate 132. Thus, providing an ink layer 130. The ink layer 130 has a first side 133 and a second side 134. The first side 133 of the ink layer 130 is attached 310 to the first surface 142 of the semi-permeable layer 140. The second side 134 of the ink layer 130 is attached 312 to the second side 122 of the double-sided adhesive 120.

The method 300 can also include providing 302 a protective layer 150 according to the method 250 for preparing the protective layer 150 and coating 304 the first surface 142 of the semi-permeable layer 140 with the protective layer 150 prior to attaching the ink layer 130 to the semi-permeable layer 140 so that the protective layer 150 is arranged between the semi-permeable layer 140 and the ink layer 130. Thus, providing an additional barrier that hinders the migration of ink 131 from the ink layer 130 to the protective layer 150 and subsequently to the food product 11 and the food package 10.

The method 300 further includes a final step of attaching 314 the first side 121 of the double-sided adhesive 120 to a barrier film 110 for a food package 10.

The indicator 100 provided as a label can be attached to a barrier film 110 for a food package 10 by integrating the indicator 100 with the barrier film 110 by a roll to roll or by mechanically pressing. Preferably, the double sided-adhesive layer 120 is protected by a liner prior application.

Further, the invention has mainly been described with reference to a few embodiments. However, as is readily understood by a person skilled in the art, other embodiments than the ones disclosed above are equally possible within the scope of the invention, as defined by the appended claims.

In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. The terms “a”. “an”. “first”. “second” etc. do not preclude a plurality. Reference signs in the claims are provided merely as a clarifying example and shall not be construed as limiting the scope of the claims in any way.

1. A food quality indicator comprising:
an ink layer comprising an ink absorbed on a porous substrate configured to indicate the presence of analytes,
an adhesive layer, wherein the adhesive layer is a double-sided adhesive layer comprising a first side and a second side,
a semi-permeable layer comprising a first surface and a second surface, wherein the semi-permeable layer is permeable to gases and impermeable to liquids,
wherein the ink layer is enclosed between the second side of the adhesive layer and the first surface of the semi-permeable layer.

2. The food quality indicator according to claim 1, wherein the analytes migrate through the semi-permeable layer to reach the ink layer.

3. The food quality indicator according to claim 1, further comprising a protective layer that is coated on the first surface of the semi-permeable layer, wherein the protective layer is configured to prevent the migration of ink from the ink layer.

4. A food quality indicator according to claim 3, wherein the protective layer is permeable to gases.

5. The food quality indicator according to claim 3, wherein the analytes migrate through the semi-permeable layer and the protective layer, to reach the ink layer.

6. A food quality indicator according to claim 3, wherein the protective layer is a hydrophobic layer.

7. The food quality indicator according to claim 3, wherein the protective layer comprises a polymer and a solvent.

8. The food quality indicator according to claim 7, wherein the polymer and the solvent of the protective layer have a weight ratio in the interval 0.01:2 to 0.2:0.4, such as 0.01:1, preferably 0.15:0.56.

9. (canceled)

10. (canceled)

11. A food quality indicator according to claim 1, wherein the semi-permeable layer is non-transparent.

12. A food quality indicator according to claim 1, wherein the semi-permeable layer is configured to be printed on.

13. (canceled)

14. A food quality indicator according to claim 1, wherein the ink layer and the semi-permeable layer have different surface areas so the ink layer and the semi-permeable layer can be fastened to the second side of the double-sided adhesive layer.

15. The food quality indicator according to claim 1, wherein the first side of the adhesive layer of the food quality indicator is configured to be attached to a barrier film of a food package.

16. The food quality indicator according to claim 1, wherein the food quality indicator is configured to be attached inside a food package containing food products producing analytes upon decomposition, such as meat, poultry, fish, dairy products, fruits vegetables, processed food.

17. The food quality indictor according to claim 1, wherein the ink comprises a surfactant, a thickening agent, a pigment indicator, a benzopyrone compound, a solvent and and a colour enhancer.

18-30. (canceled)

31. The food quality indictor according to claim 1, wherein the porous substrate of the ink layer is selected from cellulose, paper, synthetic paper, polyolefin-based synthetic material, thermoplastic copolyester (TPPE), low-density or high-density polyethylene, low-density or high-density polypropylene, nylon, nonwoven fabric, fabric or polymeric membranes.

32. The food quality indictor according to claim 1, wherein the porous substrate of the ink layer has pores with pore size between 10 μm to 100 μm, with a thickness ranging between 0.05 millimetre to 0.5 millimetre, preferably between 0.05 milometer to 0.1 millimeter.

33. A method for preparing an ink of a food quality indicator according to claim 1, the method comprises the steps of:
preparing a mixture by dissolving a surfactant in a solvent;
adding pigment coupled to silane to the mixture;
treating the mixture with ultrasonicating for 10-30 minutes followed by vortex mixing for 20-30 minutes;
adding a colour enhancer and a polymer thickening agent to the mixture;
treating the mixture by ultrasonicating the mixture for 10-30 minutes followed by vortex mixing for 20-30 minutes;
adding a coupling agent to the mixture; and
heating the mixture to 30-60° C.

34. (canceled)

35. A method for manufacturing the indicator of the food quality indicator according to claim 1, the method comprises the step of:
providing an ink according to the method for preparing the ink;
applying a layer of the ink to a porous substrate to provide an ink layer;
attaching a first side of the ink layer to a semi-permeable layer;
attaching a second side of the ink layer to a second side of a double-sided adhesive.

36. The method according to claim 35, the method further comprising the step of:
providing a protective layer according to the method for preparing the protective layer;
coating the semi-permeable layer with the protective layer prior to applying the ink layer to the semi-permeable layer so that the protective layer is arranged between the semi-permeable layer and the ink layer.

37. The method according to claim 35, the method comprising attaching the first side of the double-sided adhesive to a barrier film for a food package.
